(19) United States
(12) Patent Application Publication
(10) Pub. No.: US 2016/0008152 A1
(43) Pub. Date: Jan. 14, 2016

(54) APPARATUS, SYSTEMS AND METHODS FOR MEDICAL DEVICE EXPANSION

(71) Applicant: Abbott Cardiovascular Systems, Inc., Santa Clara, CA (US)

(72) Inventor: Michael Lee Green, Pleasanton, CA (US)

(21) Appl. No.: 14/860,402

(22) Filed: Sep. 21, 2015

Related U.S. Application Data
(62) Division of application No. 13/490,225, filed on Jun. 6, 2012.

(51) Int. Cl. A61F 2/915; B21D 39/20

(52) U.S. Cl. CPC A61F 2/915; B21D 39/20; A61F 2240/001; A61F 2210/0019

(57) ABSTRACT

A method for manufacturing a medical device. The method can include forming a medical device from a tube having a first diameter; uniformly expanding the medical device from the first diameter to a second diameter at which the medical device can be left within a body vessel, the medical device being expanded from the first diameter to the second diameter while being continuously positioned on an expander; and heat setting the expanded medical device at the second diameter while the medical device is positioned on the expander.

### CROSS-REFERENCE TO RELATED APPLICATIONS

This application is a divisional of U.S. patent application Ser. No. 13/490,225, filed 6 Jun. 2012, the disclosure of which is expressly incorporated herein by this reference.

### BACKGROUND OF THE INVENTION

I. The Field of the Invention

The present invention generally relates to the field of medical devices. More specifically, the present invention relates to methods, systems, and devices for manufacturing a self-expanding medical device.

II. Related Technology

The use of intravascular devices to treat cardiovascular diseases is well known in the field of medicine. The need for a greater variety of devices to address different types of circumstances has grown tremendously as the techniques for using intravascular devices has progressed. One type of intravascular device is a stent or scaffold. Stents and scaffolds are generally cylindrically shaped intravascular devices that are placed within an artery (or other vessel within the body) to hold it open. The device can be used to reduce the likelihood of restenosis or recurrence of the blocking of a blood vessel and can be placed within an artery on a permanent basis, such as a stent, or a temporary basis, such as a scaffold. In some circumstances, a stent or scaffold can be used as the primary treatment device where it is expanded to dilate a stenosis and left in place.

A variety of stent or scaffold designs have been developed. Examples include coiled wires in a variety of patterns that are expanded after being placed within a vessel on a balloon catheter, helically wound coiled springs manufactured from expandable heat sensitive metals, stents or scaffolds shaped in zig-zag patterns, and self-expanding stents or scaffolds inserted in a compressed state for deployment in a body lumen.

Stents and scaffolds can have various features. For instance, a stent or scaffold can have a tubular shape formed from a plurality of interconnected struts and/or legs that can form a series of interconnected rings. In the expanded condition, the stent or scaffold can have a cylindrical shape to expand in an artery. One material for manufacturing self-expanding stents or scaffolds is nitinol, an alloy of nickel and titanium.

The conventional approach to manufacture a self-expanding stent or scaffold is to begin by laser cutting the design of the stent or scaffold from a tube having a diameter that is approximately equal to the desired diameter of the compressed (i.e., unexpanded) stent or scaffold. The tube is then deburred to clean any imperfections due to the cutting. Once the tube has been deburred, the tube is then expanded to the desired diameter, which is the diameter the stent will maintain when left within a body vessel. The tube is then heat set at the desired expanded diameter to maintain the tube at that diameter.

Conventionally, expanding the stent or scaffold to the desired expanded diameter requires an iterative process: The tube is positioned on a mandrel having a diameter that is slightly larger than the diameter of the compressed tube, thereby expanding the tube. Heat is applied to the tube while the tube is on the mandrel to heat set the tube at the new diameter. The tube and mandrel are allowed to cool to complete the heat setting, and the tube is then removed from the mandrel. This process is then repeated with a slightly larger mandrel to expand the tube further. This iterative process of expanding the tube a little at a time is repeated until the desired expanded diameter is attained.

Although the conventional manufacturing approach discussed above generally yields acceptable self expanding medical devices, the approach has some shortcomings. For example, it is cumbersome and time consuming due, in large part, to the iterative heating and cooling processes. In addition, a significant amount of energy is used by heating and reheating the medical device and the mandrel during each iteration. Another shortcoming is that, in many instances, cracks are induced in the stent or scaffold during conventional manufacturing due to undesired torque, tension, expansion, and/or compression.

### BRIEF SUMMARY OF THE INVENTION

Embodiments of the invention relate to the expansion of medical devices including implantable medical devices such as stents or scaffolds.

In one embodiment, a method of manufacturing a medical device can include forming a medical device from a tube having a first diameter; uniformly expanding the medical device from the first diameter to a second diameter at which the medical device can be left within a body vessel, the medical device being expanded from the first diameter to the second diameter while being continuously positioned on an expander; and heat setting the expanded medical device at the second diameter while the medical device is positioned on the expander.

In another embodiment, a method of manufacturing a medical device can include positioning a medical device on a transport assembly having a plurality of transport mechanisms, the transport mechanisms being arranged generally parallel to a central longitudinal axis; positioning a portion of the transport assembly on an expander so that the medical device becomes positioned radially over the expander; radially expanding the medical device with the expander while the medical device is positioned on the transport assembly; and heat setting the expanded medical device while the medical device is positioned on the expander, the acts of radially expanding the medical device and heat setting the expanded medical device being performed while the medical device is positioned in a heated thermal chamber.

In another embodiment, a system for uniformly expanding and heat setting a medical device can include a thermal chamber and an expander at least partially positioned within the thermal chamber. The thermal chamber maintains the expander at a predetermined elevated temperature. The expander is configured to uniformly expand a medical device as the medical device is advanced over the heated expander and heat set the expanded medical device while the medical device is positioned on the heated expander.

In some embodiments of the invention, the medical device can be placed over a transport assembly having a plurality of transport mechanisms. The transport mechanisms can then be expanded with an expander, thereby uniformly expanding the medical device. The medical device can be expanded at any operable temperature. In some embodiments, the medical device can be expanded while within a temperature controlled zone. In some embodiments, the medical device can be heat set while in the expanded state.

The transport mechanisms may engage with corresponding transport guides, such as recesses, grooves, or channels, in the expander that keep the transport mechanisms uniformly spaced circumferentially around the expander, while the transport mechanisms provide a separation between the medical device and the expander body. As a result, the transport mechanisms can act as a transport to reduce friction that may otherwise occur between the medical device and the expander during expansion or manufacture of the medical device. By reducing friction, the medical device can be expanded with less susceptibility to adverse effects such as compression, tension, fracturing, torquing, bending, uneven expansion, and the like or any combination thereof.

A medical device can thus be expanded in one embodiment by positioning the medical device over a transport assembly that includes a plurality of transport mechanisms, such as wires. The transport mechanisms can be arranged generally parallel to a central longitudinal axis of the expander. Next, at least a portion of the transport assembly and at least a portion of the medical device can be positioned over an expander, such as a mandrel. Then, at least a portion of the medical device can be radially expanded with the expander.

The expander may have a central longitudinal axis and a body having an outer surface. The outer surface may have a plurality of longitudinal transport guides, such as wire recesses, grooves, or channels defined therein. The longitudinal transport mechanisms can be configured to be positioned at least partially within the transport guides to guide the transport assembly for translation of the transport assembly with respect to the expander, parallel to the longitudinal axis. The expander may also have a portion with a first diameter, a portion with a second larger diameter, and a transition portion that transitions the expander from the first diameter to the second diameter.

In one embodiment, the medical device can be expanded by axially translating the expander relative to the medical device. The transport mechanisms can transport the medical device by reducing friction between the medical device and the expander as the expander moves axially (or while the medical device moves axially along the expander). During heat-setting of the medical device, the medical device can be heat-set in the expanded position, for instance.

In one embodiment, the transport assembly can comprise a wire array and the transport mechanisms can comprise the wires that make up the wire array. Correspondingly, the transport guides can comprise wire guides arranged generally parallel to the central longitudinal axis of the expander so as to receive and guide the wires over the expander. The medical device can be expanded by positioning the medical device over the wires of the wire array and then moving the wire array toward the expander so that the wires are received within the wire guides of the expander. Next, at least a portion of the wire array and at least a portion of the medical device can be advanced onto the expander. The medical device can then be radially expanded by the expander as the medical device moves with the wires within the wire guides.

These and other advantages and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. In the drawings, like numerals designate like elements. Furthermore, multiple instances of an element may each include separate letters appended to the element number. For example two instances of a particular element “20” may be labeled as “20a” and “20b”. In that case, the element label may be used without an appended letter (e.g., “20”) to generally refer to every instance of the element; while the element label will include an appended letter (e.g., “20a”) to refer to a specific instance of the element.

FIG. 1 illustrates an exploded view of a system for expanding a medical device according to one embodiment;

FIG. 2 is a perspective view of the expander shown in FIG. 1;

FIGS. 3A, 3B, and 3C are cross sectional views of the expander of FIG. 2, taken along section lines 3A-3A, 3B-3B, and 3C-3C, respectively, of FIG. 2;

FIGS. 4A-4C illustrate a method for expanding a medical device using the system shown in FIG. 1, according to one embodiment;

FIGS. 5A-5C illustrate a method for deploying a medical device according to one embodiment;

FIGS. 6A-6B is a side view of an expander according to another embodiment;

FIGS. 7A-7B are cross sectional views of an expander according to another embodiment;

FIGS. 8A-8C illustrate a method for expanding a medical device using the system shown in FIG. 1, according to another embodiment;

FIG. 9 illustrates an exploded view of a system for expanding a medical device according to another embodiment;

FIG. 10 is a end view of the integrated wiring guide shown in FIG. 9;

FIGS. 11A-11B illustrate a method for expanding a medical device using the system shown in FIG. 9, according to one embodiment; and

FIGS. 12A-12B illustrate embodiments for rotationally aligning an integrated wiring guide and an expander.

### DETAILED DESCRIPTION

As used in the specification and appended claims, directional terms, such as “top,” “bottom,” “up,” “down,” “upper,” “lower,” “proximal,” “distal,” and the like are used herein solely to indicate relative directions and are not otherwise intended to limit the scope of the invention or claims.

Methods and devices are provided herein for expanding a medical device. The methods provided through the systems and devices are repeatable and reduce the possibility of incorrectly expanding medical devices during the manufacturing process. Further, the methods provided herein reduce the possibility of undesired torque, tension, expansion and compression of the stent or scaffold during manufacture.

In at least one embodiment, a method for expanding a medical device includes placing the medical device over longitudinally oriented transport mechanisms, such as wires. The medical device is then expanded while in place over the transport mechanisms. The transport mechanisms can provide a bearing-type surface to allow for even expansion while reducing the potential for deformation. In at least one embodiment, the transport mechanisms can be positioned on an expander with transport guides, such as recesses, grooves, or channels, for maintaining a desired spacing between the transport mechanisms. In at least one embodiment, the expander can cause the medical device to expand without the expander itself expanding. In other embodiments, the expander can be expanded with a separate expanding mechanism that is inserted into the expander to expand the expander, and thereby expand the transport mechanisms and the medical device. Accordingly, a variety of methods, systems, and devices can be used to expand a medical device over longitudinally oriented transport mechanisms, as will be discussed in more detail below.

Reference will now be made to figures wherein like structures will be provided with like reference designations. It is understood that the drawings are diagrammatic and schematic representations of exemplary embodiments of the invention, and are not limiting of the present invention nor are they necessarily drawn to scale.

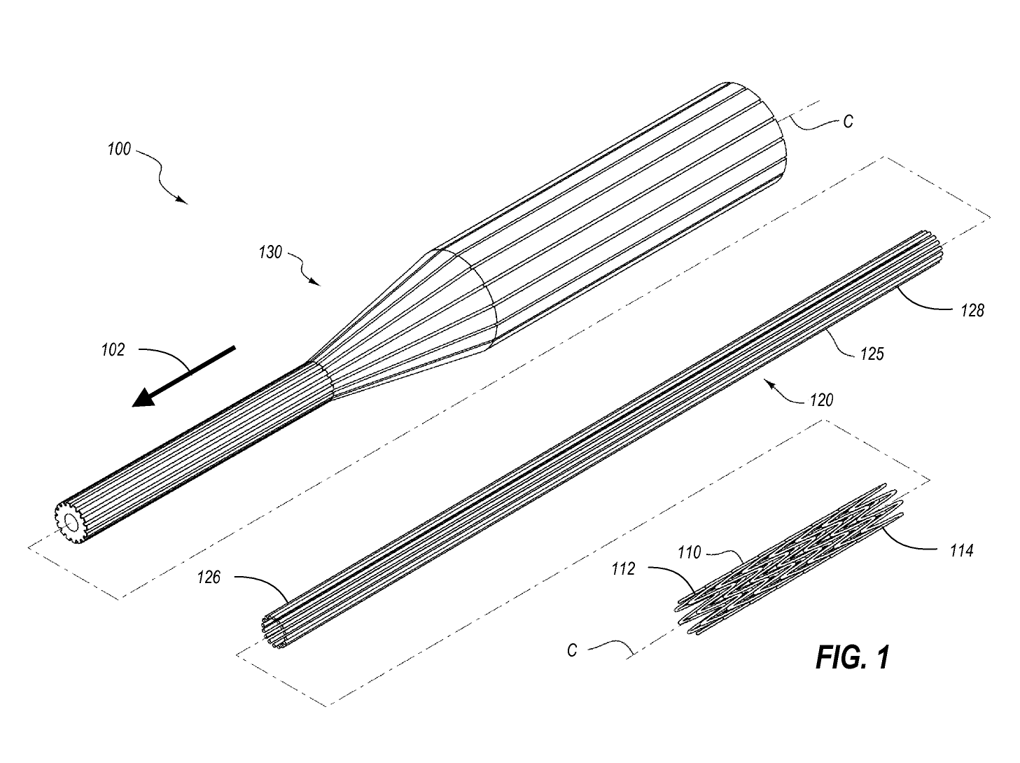

FIG. 1 illustrates one embodiment of a system 100 for expanding a medical device 110, such as a vascular device, extending between a proximal end 112 and a spaced apart distal end 114. For ease of reference, a coordinate system will be referenced in discussing system 100 (and the other systems discussed herein) that includes a central axis C. Elements that are generally parallel to central axis C will also be described as being longitudinally oriented relative to central axis C while elements that are generally transverse or perpendicular to central axis C will be described as being radially oriented relative to central axis C. In addition, the direction indicated by arrow 102 that is parallel to central axis C will be referred to as the “proximal” direction and the opposite direction will be referred to as the “distal” direction. As such, movement in the proximal and distal directions may be referred to as proximal and distal movement, respectively.

In the illustrated embodiment, system 100 includes a longitudinally oriented transport assembly in the form of a wire array 120 having a plurality of individual transport mechanisms in the form of wires 125 extending from a proximal end 126 to a distal end 128. The number of wires can vary, as discussed below. The wires can be made of metals or alloys, such as, but not limited to, stainless steel, titanium, tantalum, tungsten, or alloys thereof, nickle chromium (commonly known as nichrome) quartz, glass, glass thread, polymers, or other high temperature material. Using wires that can sustain high temperatures allows the medical device to be heat treated (e.g., such as shape set using heat).

Although reference has been made to the use of wires and a wire array, respectively, as the transport mechanisms and transport assembly, one skilled in the art will appreciate that other structures can also perform the functions of the transport mechanisms and transport assembly. For example, and not by way of limitation, other structures that can be used as the transport mechanisms include strips, ribbons, yarns, threads, rods, or other structures having the desired strength and rigidity, with associated flexibility and resiliency to allow the structure to i) provide a bearing-type surface for the medical device, ii) separate at least a portion of the medical device from an expander or expander mechanism during a manufacturing process, or iii) otherwise perform other functions described or identified from the description contained herein.

Wire array 120 is configured to receive medical device 110 thereon. System 100 also includes an expander 130, which can also be described as a mandrel in the present embodiment. Expander 130 includes features that are configured to guide and/or partially receive wire array 120.

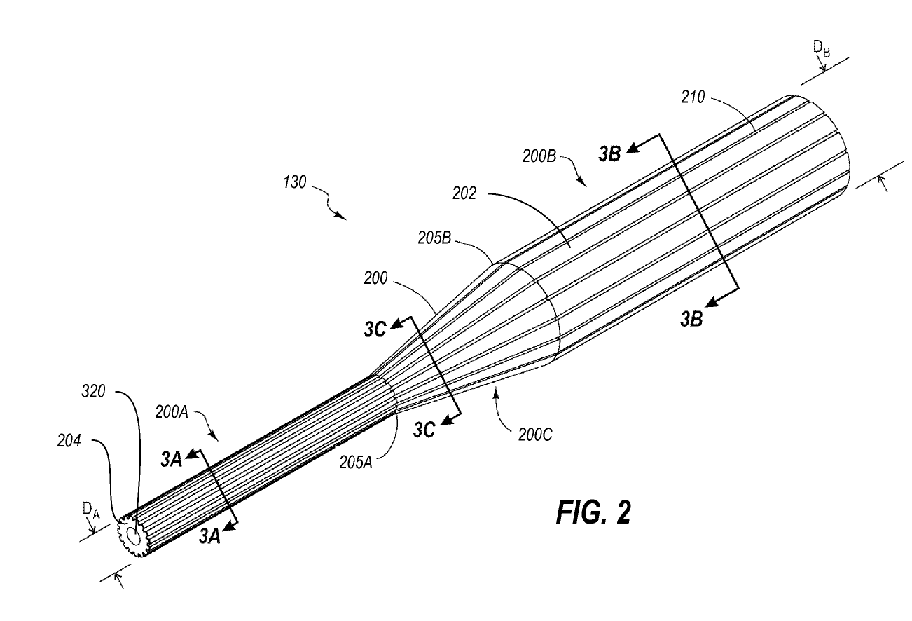

For example, expander 130 illustrated in FIG. 2 generally includes a body 200 having an outer surface 202 extending between a proximal end face 204 on a proximal end 200A and a distal end 200B. Body 200 can have a first diameter DA at proximal end 200A that transitions to a second diameter DB that is greater than the first diameter DA. In at least one embodiment, the second diameter DB is at distal end 200B, though it will be appreciated that the first diameter DA can transition to any number of varying diameters at any number of locations between proximal end 200A and distal end 200B.

In at least one embodiment, proximal end 200A and distal end 200B can each be as long or longer than medical device 110 (FIG. 1) which system 100 (FIG. 1) is configured to expand. Such a configuration can provide support for medical device 110 both before and after medical device 110 is expanded as the diameter of the portion of body 200 is increased. As the diameter of body 200 increases, body 200 continues to support wire array 120 which, in turn, supports and provides a bearing-type surface for medical device 110 (FIG. 1).

In the illustrated embodiment, body 200 transitions from the first diameter DA to the second diameter DB at a transition portion 200C. The depicted transition portion 200C includes a ramped profile with a shoulder portion 205A associated with proximal end 200A and a shoulder portion 205B associated with distal end 200B. As such, transition portion 200C is substantially frustoconically shaped in the depicted embodiment. It will be appreciated, however, that other shapes are possible and that transition portion 200C and shoulder portions 205A, 205B can have any profile, and that any number of transition portions can be provided.

In the depicted embodiment, the diameters of proximal and distal ends 200A and 200B remain substantially unchanged along the lengths of the respective end. That is, the diameter DA of proximal end 200A remains substantially unchanged along the entire length of proximal end 200A and the diameter DB of distal end 200B remains substantially unchanged along the entire length of distal end 200B. However, if desired the diameter of proximal end 200A and/or the diameter of distal end 200B can instead vary along the length of the corresponding end. For example, in one embodiment, the diameter DB of distal end 200B progressively increases as distal end 200B extends distally away from transition portion 200C. In that embodiment, distal end 200B can have a ramped profile similar to transition portion 200C. That embodiment can be used to expand and heat set medical devices into a tapered heat set configuration, such as, e.g., a tapered stent.

To correspond to wires 125, transport guides in the form of wire guides 210 are defined on outer surface 202 of body 200 and are distributed circumferentially about outer surface 202. Each wire guide 210 extends longitudinally between end face 204 at proximal end 200A, through transition portion 200C, and toward distal end 200B. Wire guides 210 are configured to receive wires 125 of wire array 120 (FIG. 1). Such a configuration can constrain wires 125 and keep them in a particular spatial orientation during expansion of medical device 110. As such, the number of wire guides 210 should be equal to or greater than the number of wires 125 in wire array 120. Ideally, the number of wire guides 210 equals the number of wires 125.

Further, wire guides 210 can control friction between medical device 110 and body 200 of the expander 130 during expansion. For instance, changing the depth of wire guides 210 changes the height of wires 125 extending above surface 202 of body 200 to function and provide a bearing-type surface upon which at least a portion of medical device 110 (FIG. 1) rests. With more of each wire 125 exposed above surface 202, there is a decreased likelihood that a portion of medical device 110 (FIG. 1) frictionally engages body 200 upon axial movement of medical device 110 (FIG. 1) relative to body 200, or vice versa.

This limits the possibility of unwanted frictional contact that could damage medical device 110 (FIG. 1) due to the application of undesired torque, tension, expansion, and/or compression to medical device 110 (FIG. 1) during the manufacturing processes. In one configuration, therefore, wires 125 extend radially outward from wire guides 210 sufficiently to prevent contact between medical device 110 (FIG. 1) and the body 200. In another configuration, wires 125 extend radially outward from wire guides 210 sufficiently to prevent contact that would be sufficient to damage medical device 110 (FIG. 1).

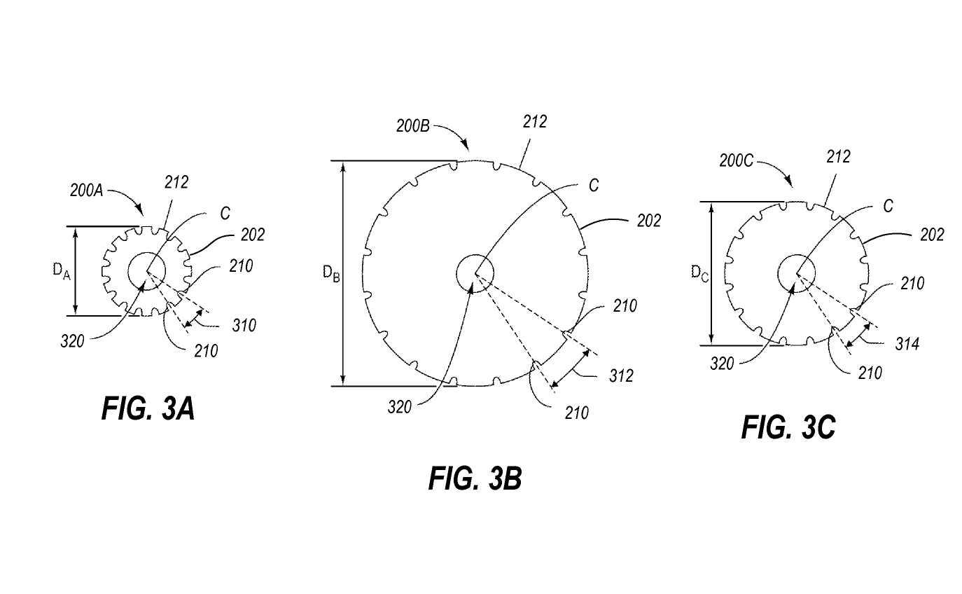

Transition portion 200C may have a tapered configuration with a slope that allows expansion of medical device 110 to proceed smoothly without unduly expanding a portion of medical device 110 relative to an adjacent portion of the medical device. Further, the cross sectional shape of expander member 130 is typically similar in all portions and thus the expansion of medical device 110 can be to the same shape. For example, as shown in FIGS. 3A-3C, the cross sectional shape of expander 130 in the depicted embodiment is generally circular for each portion.

In alternative embodiments, the portions of expander 130 may have different cross sectional shapes. This may allow, for example, a medical device to be expanded from a circular cross section to some other cross-sectional shape, such as, but not limited to, an oval cross section, a polygonal cross section, or some other regular or irregular geometric cross section. In addition, the expander 130 can be shaped to accommodate the shape of an anticipated deployment site. As a result, the final cross sectional area of the medical device can vary. Also, the cross sectional shape can vary as well.

Expander 130 can be fabricated from a variety of different materials. For instance, expander 130 can be made from metals, alloys, plastics, polymers, composites, ceramics, or any combinations thereof. Expander 130 can alternatively be made of other materials, as desired, based upon the particular medical device being formed and the temperatures and/or pressures that expander 130 is to withstand during the manufacture of the medical device. In another configuration, expander 130 can be plated with another material, such as, but not limited to, a chromium coating or a diamond chromium coating, such as Armoloy®, or a nickel-phosphor alloy, such as NEDOX® 10K™-1 or MAGNAPLATE HMF, both manufactured by General Magnaplate Corporation. In one embodiment, expander 130 can be fabricated from stainless steel or a nickel titanium alloy, such as nitinol. In various embodiments, the materials forming expander 130 can withstand a temperature from about 250° C. to about 600° C., from about 250° C. to about 650° C., from about 300° C. to about 600° C., from about 300° C. to about 550° C., from about 450° C. to about 600° C., from about 450° C. to about 550° C., or some other range known to one skilled in the art in view of the teachings contained herein.

FIG. 3A illustrates a cross-sectional view of proximal end 200A of expander 130 taken along section line 3A-3A of FIG. 2, FIG. 3B illustrates a cross-sectional view of distal end 200B taken along section line 3B-3B, and FIG. 3C illustrates a cross-sectional view of transition portion 200C taken along section line 3C-3C of FIG. 2.

As shown particularly in FIG. 3A, proximal end 200A has a generally circular or tubular cross sectional profile with the first diameter DA. A guide opening or lumen 320 can longitudinally extend into expander 130 from proximal end face 204, if desired, and extend at least partially through expander 130, as shown in FIGS. 3A-3C, although this is not required. In at least one embodiment, guide opening 320 is centered on central axis C of the body. Guide opening 320 can be used to guide wire array 120 and medical device 110 onto the expander 130, as discussed below.

Wire guides 210 are positioned circumferentially about outer surface 202 of body 200 (FIG. 2) and about proximal end 200A. In at least one embodiment, each wire guide 210 can include a profile that is partially circular, although various other profiles are possible which receive wire 125.

Wire guides 210 are separated by angular separations 310 relative to central axis C. In the depicted embodiment, the angular separations between the individual wire guides are substantially equal, but this is not required. In other embodiments, the angular separations can be different and wire guides 210 can be arranged in a manner that is partially symmetrical or asymmetrical.

Each wire guide 210 can have any desired depth and dimension and shape. In at least one embodiment, each wire guide 210 can include a recess, groove, or channel having a generally hemispherical inner portion. In other embodiments, the recess, groove, or channel can be square shaped, angular, and the like. Further, each recess, groove, or channel can have an inner portion having a central angle of any size. Finally, the arrangement of the wire guides provides, in one embodiment, a spline-type geometry to keep the wires uniformly spaced circumferentially around the expansion member. For instance, adjacently positioned wire guides 210 can be separated by a portion of body 200 or a spline 212 as illustrated in FIG. 3A.

For ease of reference, positions of wire guides 210 relative to other elements will be described with reference to the central portion of the recesses defining wire guides 210. It will be appreciated that other reference points can be used to describe relative positions. In at least one embodiment, wire guides 210 are positioned about the perimeter of proximal end 200A such that angular separations 310 are substantially equal.

As illustrated in FIG. 3B, distal end 200B of body 200 (FIG. 2) also has a generally circular or tubular cross sectional profile, but with the second diameter DB. As previously discussed, the second diameter DB is greater than the first diameter DA. Further, at or near distal end 200B, wire guides 210 can be separated by angular separations 312. In at least one embodiment, angular separations 312 can be substantially equal with respect to distal end 200B. Further, in at least one embodiment angular separations 312 can be substantially equal to angular separations 310 between wire guides 210 on proximal end 200A (FIG. 3A). Accordingly, angular separations 312 can remain substantially constant while the diameter of body 200 (FIG. 2) increases.

The diameter of body 200 increases from the first diameter DA to the second diameter DB through transition portion 200C. As illustrated in FIG. 3C, transition portion 200C provides an angular separation 314 that is substantially similar to angular separations 310, 312 respectively associated with proximal end 200A (FIG. 3A) and distal end 200B (FIG. 3B) while increasing the diameter of body 200 (FIG. 2). Such a configuration can allow wire guides 210 to maintain wires 125 (FIG. 1) of wire array 120 (FIG. 1) evenly distributed, which can evenly distribute forces exerted by the wires during an expansion process.

Although the above-described embodiment includes evenly distributed wire guides 210, one skilled in the art will appreciate that in other configurations wire guides 210 may be unevenly distributed along all or a 200. For portion of the length of body instance, in another embodiment, the angle of angular separation 310 of wire guides 210 at at least a portion of proximal end 200A can be smaller than the angle of angular separation 312 at at least a portion of distal end 200B. Similarly, in another embodiment, the angle of angular separation 310 of wire guides 210 at at least a portion of proximal end 200A can be greater than the angle of angular separation 312 at at least a portion of distal end 200B. It will be understood that various other combinations of angular separations are also possible and known to those skilled in the art in view of the teaching contained herein.

Various methods of operation will be discussed below. It will be appreciated that when discussing movement of elements with respect to each other, either element can move while the other is stationary, or both elements can move. For example, if element A is said to move distally toward element B, this means that i) element A can move in the distal direction while element B remains stationary, ii) element B can move in the proximal direction while element A remains stationary, or iii) both elements can move toward each other.

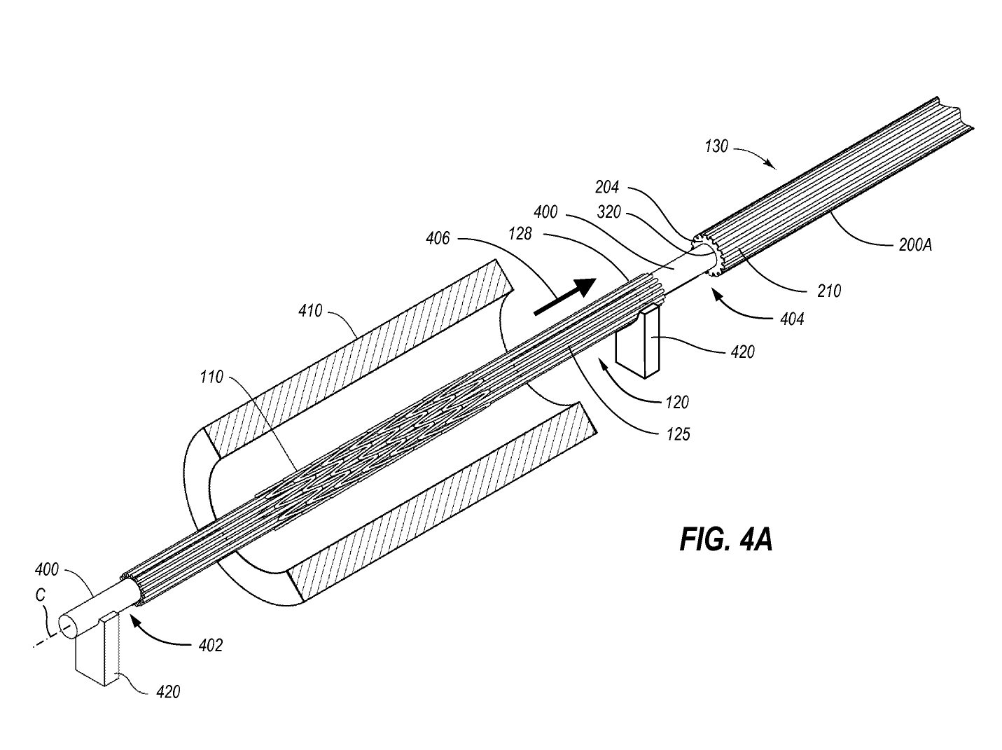

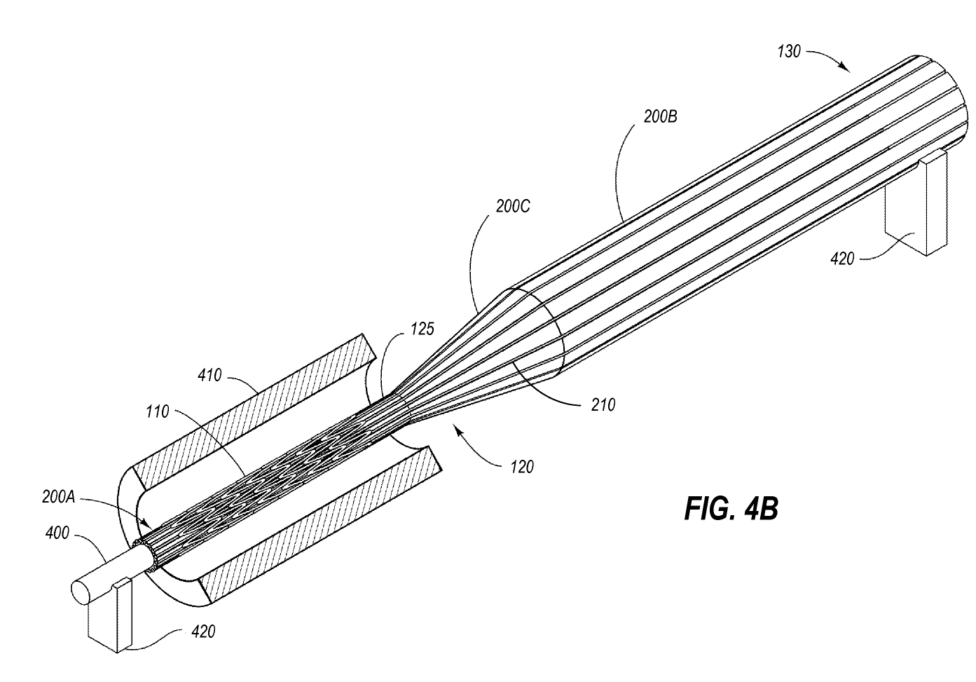

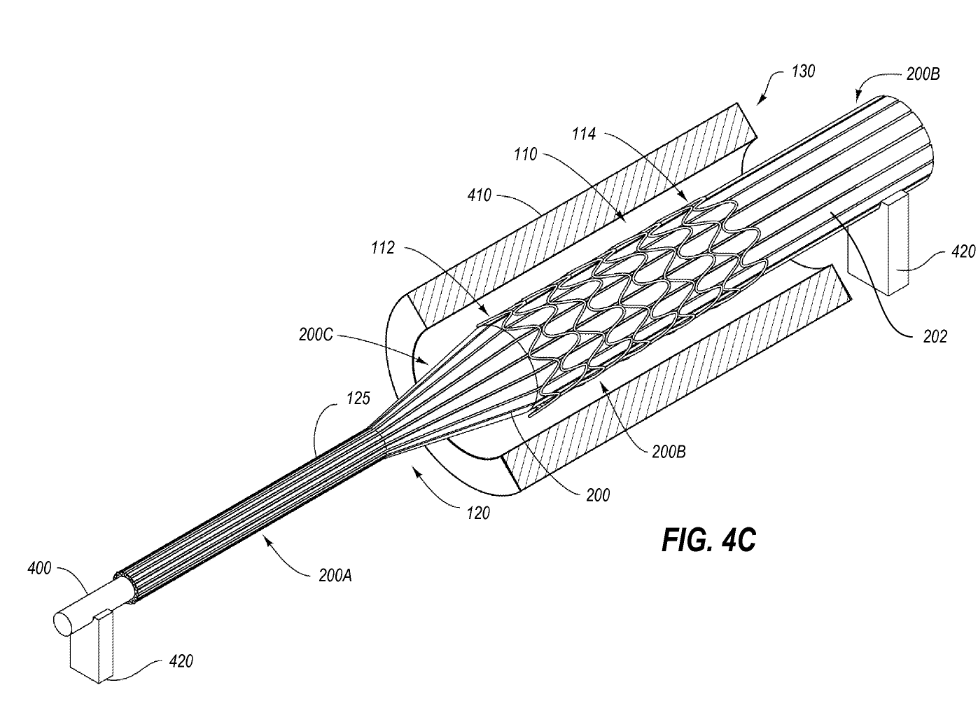

FIGS. 4A-4C illustrate one embodiment of a method of uniformly expanding medical device 110 using an axial guide 400 to move wire array 120 with respect to expander 130. Axial guide 400 extends between a proximal end 402 and a distal end 404 and is sized to be insertable into guide opening 320 in expander 130. As such, axial guide 400 can have a substantially uniform cross sectional shape along its

As with the other method embodiments described herein, while an exemplary order of steps will be described in expanding the medical device, it will be appreciated that the steps may be performed in different orders, that additional steps may be included, and/or that steps may be omitted.

Before expanding a medical device, the medical device must first be initially cut out or otherwise formed. For example, the medical device can be laser cut from a tube having a diameter that is approximately equal to the desired diameter of the compressed (i.e., unexpanded) medical device. The tube can then be deburred to clean any imperfections due to the cutting. Other initial forming methods may also be used.

As shown in FIG. 4A, the expansion process can begin by positioning wire array 120 over axial guide 400. Thereafter, medical device 110, such as, e.g., a stent or scaffold as shown in the depicted embodiment, can be positioned over wire array 120, and, consequently, over axial guide 400. In one embodiment, proximal end 126 of wire array 120 is attached or otherwise coupled to axial guide 400. In another embodiment, axial guide 400 includes a stop portion (not shown) that extends radially outward so that the proximal end 126 of wire array 120 butts up against the stop portion, thereby causing wire array 120 to move axially as axial guide 400 moves. In another embodiment, wire array 120 is not attached to axial guide 400 and is moved independent of axial guide 400.

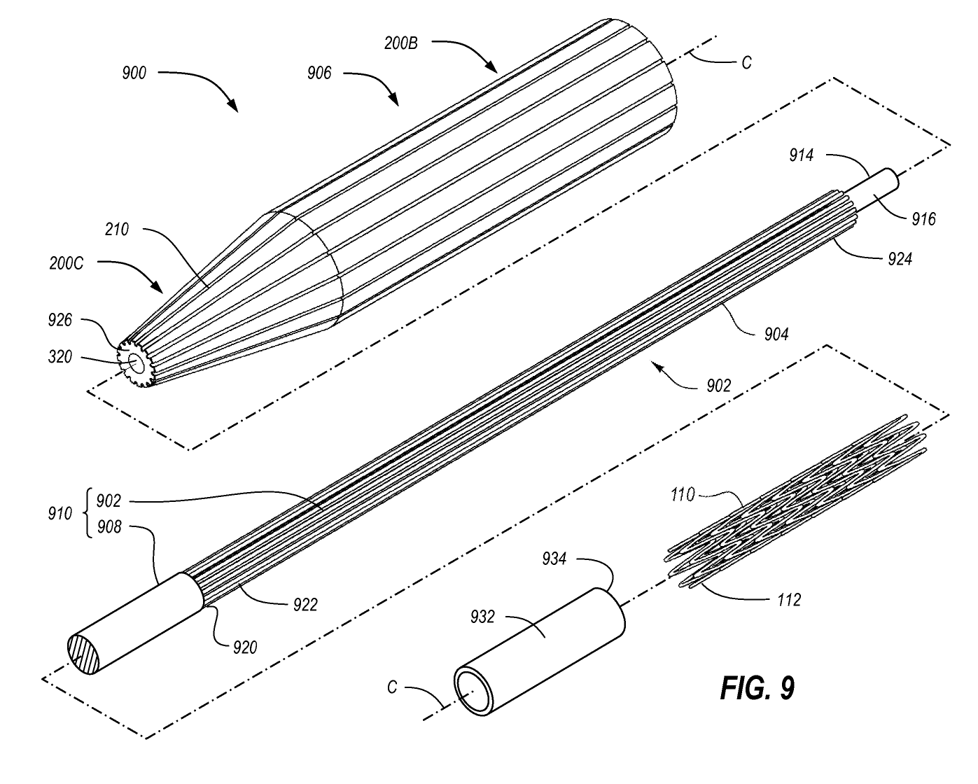

Once wire array 120 and medical device 110 are in position over axial guide 400 as shown in FIG. 4A, medical device 110 can then be positioned over proximal end 200A of expander 130. To do this, expander 130 can be positioned on axial guide 400 by way of guide opening 320. That is, axial guide 400 can be received into guide opening 320 of expander 130 at proximal end face 204 so that wire array 120 and medical device 110 can be advanced along axial guide 400 in the distal direction, denoted by arrow 406, toward expander 130. In one embodiment, wire array 120 and medical device 110 are moved distally by distal movement of axial guide 400. In another embodiment, a separate pushing mechanism can be used to advance medical device 110 and/or wire array 120 distally (see, e.g., advancement mechanism 932 in FIG. 9).

As wire array 120 and medical device 110 are distally advanced, distal end 128 of wire array 120 arrives at proximal end face 204 of proximal end 200A of expander 130. Thereafter, distal ends 128 of at least some of wires 125 can be positioned on wire guides 210 formed on proximal end 200A of expander 130. Wire array 120 and medical device 110 can then be advanced further distally so that wires 125 slide distally along wire guides 210.

As shown in FIG. 4A, medical device 110 can be positioned within a thermal chamber 410, such as an oven, a refrigerator, or any other device or apparatus configured to regulate thermal chamber 410 at one or more desired temperatures. The desired temperature(s) is/are whatever temperature(s) facilitate expansion and heat setting of the medical device. This can be affected by the material of the medical device among other factors. Thermal chamber 410 can be configured to maintain medical device 102 at a same predetermined temperature throughout preheating, expansion, and heat setting of medical device 102 or to heat medical device 102 to different predetermined temperatures for two or three of the steps. In one embodiment, the thermal chamber can be configured to heat medical device 110 to between about 450° C. to about 600° C. Of course, other temperature ranges can also be used.

Thermal chamber 410 can have an axial length that is substantially equal to or slightly longer than medical device 110. As such, thermal chamber 410 can remain axially aligned with respect to medical device 110 (i.e., thermal chamber 410 can move proximally or distally with medical device 110) so that the medical device remains positioned within thermal chamber 410 as medical device 110 and expander 130 are moved with respect to each other. For ease of reference, thermal chamber 410 will be described herein as a heating device that heats medical device 110 and expander 130 and is illustrated schematically and in cross-section.

In at least one embodiment, axial guide 400 can be supported by supports 420 that maintain axial guide 400 and/or wire array 120 radially aligned relative to thermal chamber 410. Supports 420 can allow axial guide 400 to move proximally and distally along central axis C, thereby allowing the elements that axial guide 400 is supporting to be moved into desired positions within thermal chamber 410.

Supports 420 can allow axial guide 400 to proximally and distally move medical device 110, wire array 120, and/or expander 130 into and out of thermal chamber 410. If required, supports 420 can be moved radially away from axial guide and/or wire array 120 during axial movement of wire array 120 and/or medical device 110 to allow wire array 120 and/or medical device 110 to pass.

Once wire array 120 and medical device 110 are positioned in thermal chamber 410, medical device 110 can be preheated by thermal chamber 410 to a desired temperature.

Once medical device 110 is preheated to the desired temperature, the distal advancement of wire array 120 and medical device 110 can continue until medical device 110 becomes positioned on proximal end 200A of expander 130, as illustrated in FIG. 4B. The shape and configuration of wire guides 210 help retain wires 125 in position relative to expander 130. This configuration can allow wire guides 210 to guide wires 125 as wires 125 move distally over expander 130.

As shown in FIG. 4B, thermal chamber 410 can move distally with medical device 110 so that thermal chamber 410 remains longitudinally aligned with medical device 110 and can continue heating medical device 110.

With medical device 110 preheated to the desired temperature, wire array 120 and medical device 110 can be advanced further distally by axial guide 400. As shown in FIG. 4C, as wire array 120 and medical device 110 advance distally, increasingly larger diameters of transition portion 200C of expander 130 urge wires 125, which are positioned within wire guides 210, radially outward. As wires 125 are urged radially outward, wires 125, in turn, urge medical device 110 uniformly radially outward beginning with distal end 114 of medical device 110.

Wires 125 act as a bearing-type surface that supports and guides medical device 110 while maintaining a separation between medical device 110 and expander 130. In this manner, wires 125 help reduce frictional engagement between medical device 110 and expander 130. As a result, the likelihood is reduced of medical device damage from excessive stresses associated with induced torque, tension, compression and/or expansion of the medical device during manufacture

As medical device 110 passes distally through transition portion 200C of expander 130, distal end 114 of medical device 110 becomes supported on distal end 200B of expander 130. As medical device 110 continues to move distally, proximal end 112 of medical device 110 is also uniformly expanded by the cooperation of transition portion 200C, expander 130, and wires 125 until proximal end 112 also becomes supported on distal end 200B of expander 130, as shown in FIG. 4C.

At this point, medical device 110 is fully expanded to the second diameter D_{B} (FIG. 3B) and is supported at that diameter on distal end 200B of expander 130. In the expanded configuration, medical device 110 generally takes on the shape of distal end 200B. For example, in the depicted embodiment, distal end 200B is substantially cylindrical, thereby causing medical device 110 to also have a substantially cylindrical shape in the expanded configuration. Alternatively, if a tapered medical device is desired, an expander can be used in which the diameter DB of distal end 200B progressively increases as distal end 200B extends distally away from transition portion 200C, as discussed above. Because of the changing shape of distal end 200B, medical device 110 is caused to have a tapered shape in the expanded configuration. Other expanded medical device shapes can also be obtained by using expanders having distal ends with corresponding shapes.

As shown in FIG. 4C, thermal chamber 410 can continue to move distally with medical device 110 so that thermal chamber 410 remains longitudinally aligned with medical device 110 when medical device 110 is expanded and can continue heating medical device 110. As discussed above, during expansion of medical device 110, thermal chamber 410 can maintain medical device 110 at the same predetermined temperature as during preheating, or can cause medical device to be heated to a different predetermined temperature.

Medical device 110 can remain within thermal chamber 410 after the expansion process, if desired. To do so, thermal chamber 410 can remain axially aligned with medical device 110 when medical device 110 is in the expanded configuration, as shown in FIG. 4C. In one embodiment, the expanded medical device 110 can remain within thermal chamber 410 for a predetermined period of time to heat set medical device 110 in the expanded configuration. As discussed above, during heat setting of medical device 110, thermal chamber 410 can maintain medical device 110 at the same predetermined temperature as during preheating and/or expansion or can cause medical device to be heated to a different predetermined heat setting temperature.

In at least one embodiment, while in position on expander 130, wires 125 may extend only slightly above outer surface 202 of body 200. Such a configuration may cause medical device 110 to contact outer surface 202 of expander 130 as well as wires 125 during the expansion process. Alternatively, wires 125 may extend sufficiently above outer surface 202 of body 200 so that only wires 125 contact medical device 110 during expansion while wires 125 are held in place by wire guides 210.

By substantially limiting contact of medical device 110 to only wires 125, frictional forces can be reduced compared to those generated through contact between medical device 110 and expander 130. This reduces the likelihood that medical device 110 will frictionally bind with expander 130 during heat setting or become damaged due to excessive torque, tension, expansion, and/or compression.

Further, the interaction between wires 125 and expansion member 130 can help ensure that expander 130 tracks a path that is generally parallel to central axis C as expander 130 expands medical device 110. Tracking a generally parallel path can in turn help provide even stress distribution of the stresses induced by the interaction of medical device 110 and expander 130. This even stress distribution also reduces the likelihood of medical device damage due to excessive torque, tension, expansion, and/or compression.

Once the heating and expansion process is complete, medical device 110 can be removed from heating chamber 410 and expander 130 and wire array 120. In one embodiment, medical device 110 can be removed from heating chamber 410 by essentially reversing the process discussed above. That is, axial guide 400 can be axially moved in the opposite direction (i.e., proximally), thereby moving wire array 120 and medical device 110 away from expander 130 until wire array 120 and medical device 110 are separated from expander 130. In one embodiment, the distal end 128 of wire array 120 can remain engaged with expander 130 after the expanded medical device 110 has become separated from expander 130.

As discussed above, in at least one embodiment, medical device 110 can be expanded and heat set using the method discussed above. In this embodiment, because of the heating and expansion process, the medical device is unconstrained in the expanded position. The medical device can then be constrained prior to deployment.

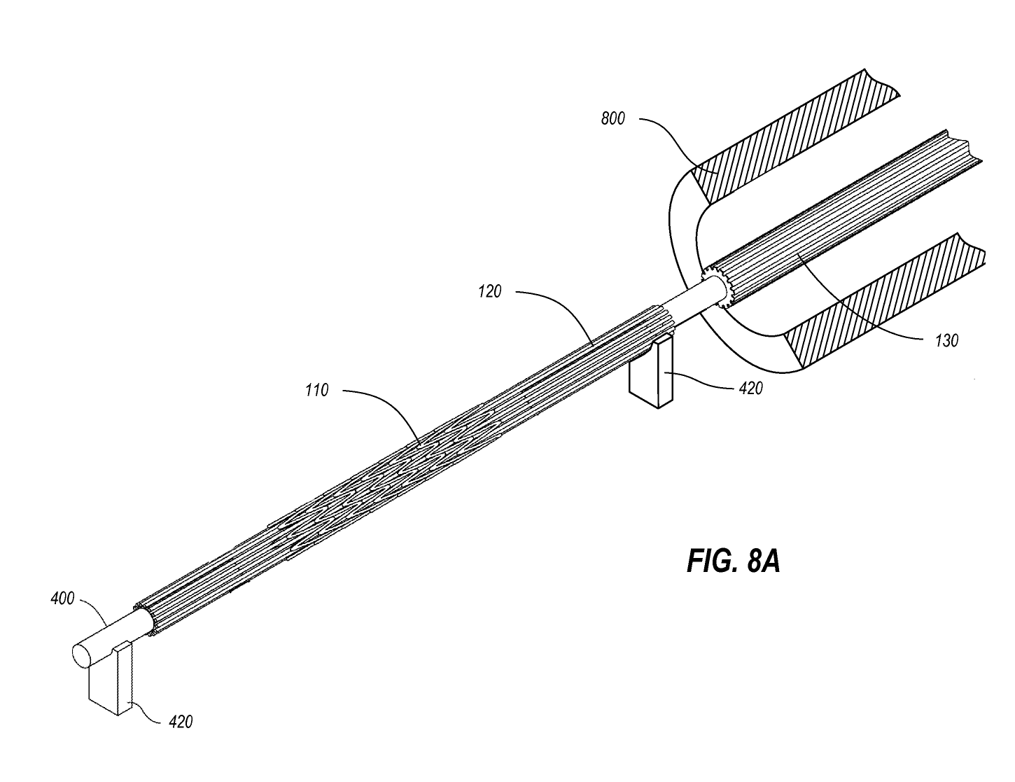

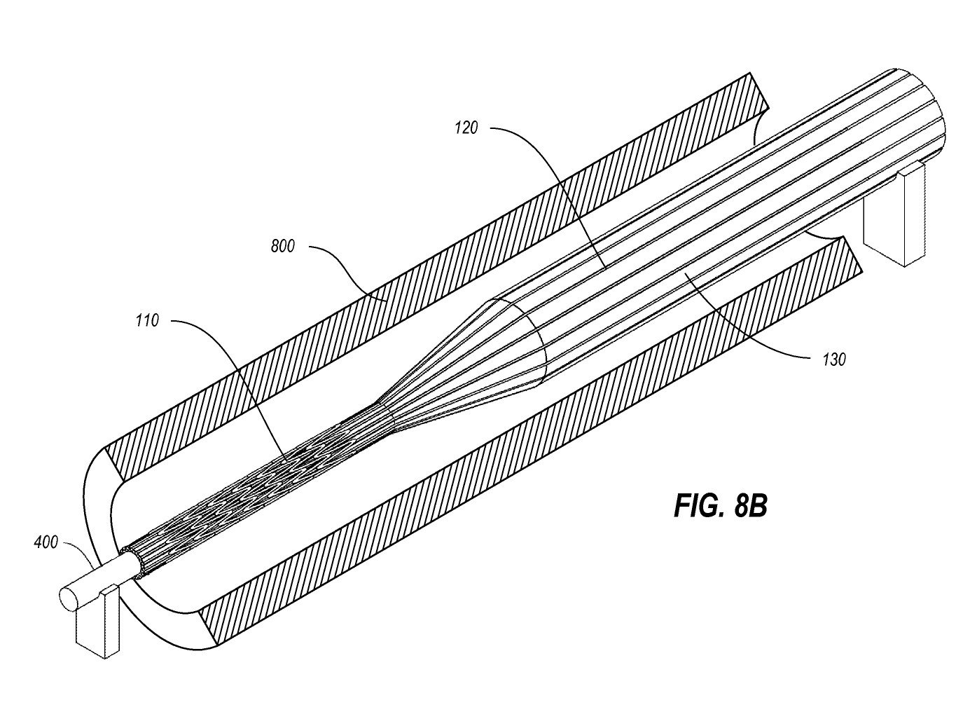

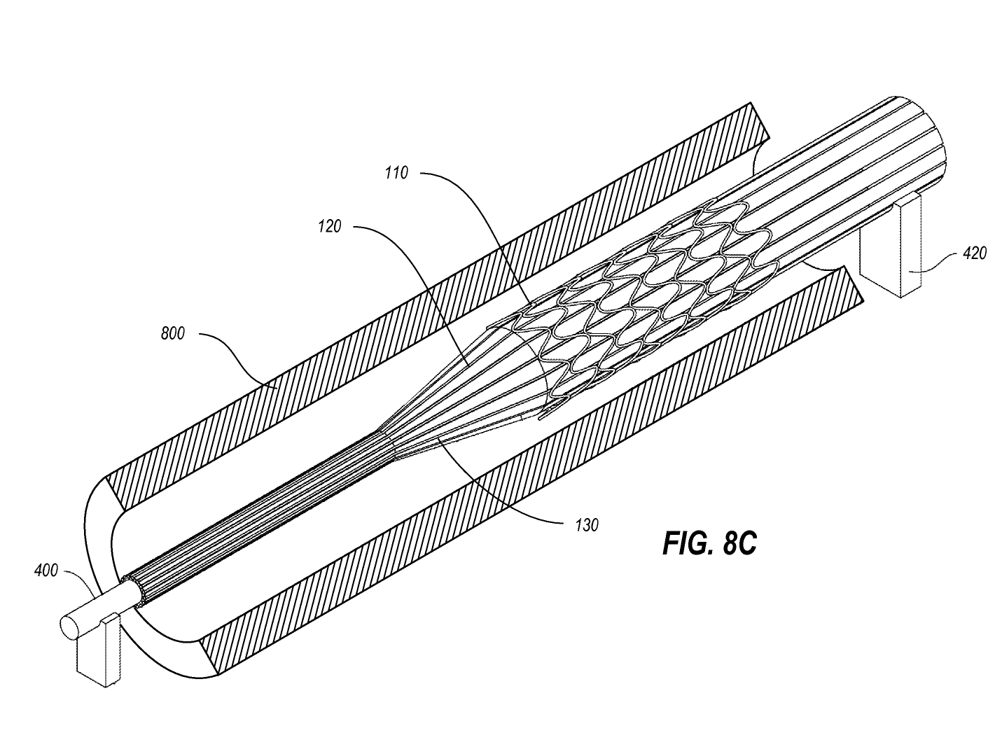

FIGS. 8A-8C illustrate another embodiment of a method of uniformly expanding (and heat setting, if desired) medical device 110 using a wire array. The method illustrated in FIGS. 8A-8C is similar to the method discussed above with respect to FIGS. 4A-4C. However, in the alternative method, a thermal chamber 800 is used having a different configuration than thermal chamber 410. Specifically, instead of having an axial length substantially equal to or slightly longer than medical device 110, thermal chamber 800 has an axial length that is substantially the same as or slightly longer or shorter than expander 130, as shown in FIGS. 8B and 8C.

As a result, thermal chamber 800 can remain axially aligned with respect to expander 130 (i.e., thermal chamber 800 can remain fixed with expander 130 or move proximally or distally with expander 130) instead of axially moving with medical device 110. This allows expander 130 and medical device 110 to both remain positioned within thermal chamber 800 when medical device 110 is mounted on expander 130, even as medical device 110 advances on expander 130.

In a similar manner to the method discussed above, the process can begin by positioning wire array 120 over axial guide 400, then positioning medical device 110 over wire array 120 and advancing axial guide 400 distally toward expander 130, as shown in FIG. 8A. In a similar manner to the method discussed above, wire array 120 and medical device 110 can be advanced distally on expander 130 to pre-heat and then expand and heat-set medical device 110, as shown in FIGS. 8B and 8C. Similar to the method discussed above, medical device 110 can remain within thermal chamber 800 during the expansion process and for a predetermined period of time thereafter, if desired, to heat set the expanded medical device. To do this, however, thermal chamber 800 does not need to move with medical device 110, but can remain axially aligned with expander 130 by remaining stationary as medical device 110 moves distally.

Whether one uses the shorter thermal chamber 410 or the longer thermal chamber 800 is generally a matter of design choice. In some aspects, longer thermal chamber 800 may provide some benefits over shorter thermal chamber 410. For example, when using thermal chamber 800, expander 130 can be maintained within thermal chamber 800 during the entire expansion process. As a result, once expander 130 is heated to a desired temperature by thermal chamber 800, the temperature of expander 130 can be maintained at a substantially constant value, such as a predetermined heat setting value of medical device 110, even between uses. Because of this, no time is lost waiting for expander 130 to subsequently heat up each time a different medical device 110 is to be expanded and heat set.

In contrast, when using shorter thermal chamber 410, different portions of expander 130 may cool and require a finite amount of time to become re-heated each time a medical device needs to be expanded and heat set due to the axial movement of thermal chamber 410 with medical device 110. This can result in delays when expanding and heat setting multiple medical devices. However, thermal chamber 410 may require less energy than thermal chamber 810 due to the shorter length. It is appreciated that other lengths can also be used for the thermal chamber, if desired.

FIG. 9 illustrates another embodiment of a system 900 for uniformly expanding (and heat setting, if desired) medical device 110. Similar to system 100, system 900 includes a wire array 902 having a plurality of wires 904 configured to be advanced onto an expander 906. However, instead of using a separate axial guide to advance the wires, system 900 combines wire array 902 and an axial guide 916 along with an advancement guide 908, to form an integrated advancement guide assembly 910. System 900 also includes an advancement mechanism 932 to aid in advancing medical device 110 over advancement guide assembly 910 and onto expander 906.

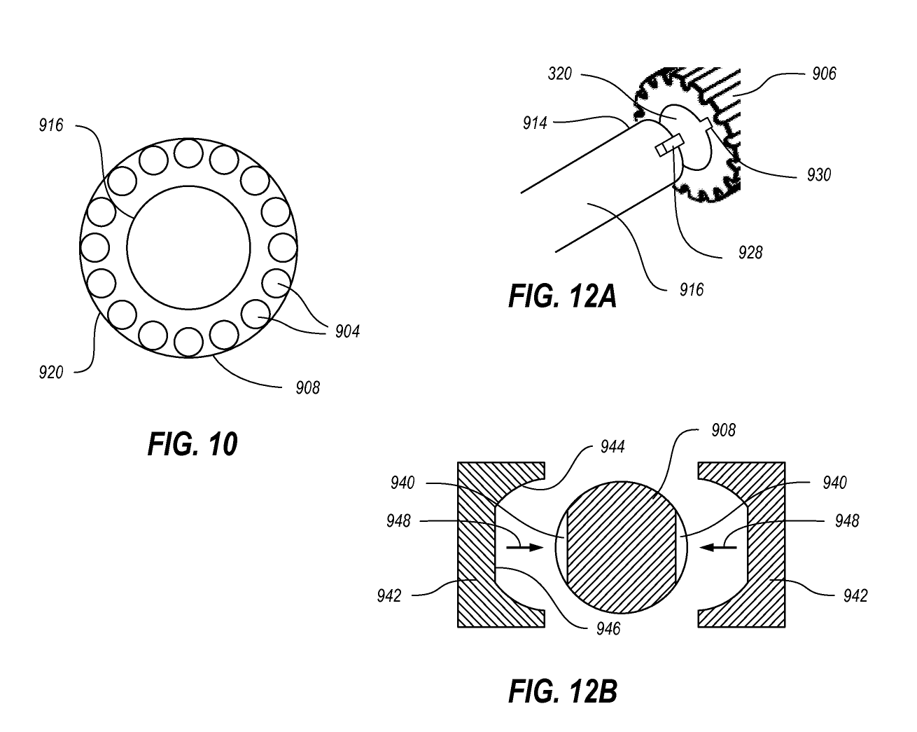

As shown in FIGS. 9 and 10, advancement guide 908 comprises a rod or the like that extends distally to a distal facing end face 920. Similar to axial guide 400, axial guide 916 is sized to be insertable into a guide opening within expander 906. Axial guide 916 extends distally from end face 920 of advancement guide 908 and has a smaller cross sectional area than advancement guide 908, as particularly shown in FIG. 10. Also as shown in FIG. 10, axial guide 916 generally extends from the center of end face 920, although this is not required. Axial guide 916 can be integrally formed with advancement guide 908 or rigidly attached thereto, such as by welding, adhesive, or any other attaching method known in the art. In some embodiments, axial guide 916 can be omitted, if desired.

As shown in FIG. 9, wire array 902 extends between a proximal end 922, positioned at end face 920, and a distal end 924. At proximal end 922, each wire 904 is welded or otherwise attached to advancement guide 908 at end face 920 to form advancement guide assembly 910. Alternatively, each wire 904 can be integrally formed with advancement guide 908 at end face 920. The number of wires in wire array 902 can vary. Although sixteen wires 904 are shown in FIG. 10, other numbers of wires can alternatively be used. For example, eight, ten, twelve, or any other number of wires can be used. However, for each wire, a corresponding wire guide should be found in the expander to receive the wire.

As shown in FIG. 10, wires 904 of wire array 902 can be positioned radially about end face 920 so that the radially outer-most edge of each wire 904 is axially aligned with the outer surface of advancement guide 908 at end face 920. That is, the diameter of wire array 902, taken at the radially outermost portion of wires 904 can be substantially equal to the diameter of advancement guide 908. By doing so, a smooth transition can be formed between advancement guide 908 and wire array 902, allowing easy passage between the two for medical device 110. Wires 904 can also be configured to radially encircle axial guide 916, if axial guide 916 is used. As such, wires 904 can longitudinally align with wire guides 210 positioned on expander 906.

In the embodiment depicted in FIGS. 9 and 10, axial guide 916 is longitudinally longer than wire array 902 and thus extends beyond distal end 924 of wire array 902. In other embodiments, wire array 902 is longer than axial guide 916 and thus extends beyond axial guide 916. In still other embodiments, axial guide 916 is not included in advancement guide assembly 910. In one embodiment, the axial guide projects from expander 906 to be received by advancement guide assembly 910.

Advancement mechanism 932 can be used to advance medical device 110 over advancement guide 908 and wire array 902 of advancement guide assembly 910 and onto expander 906. As such, advancement mechanism 932 can be substantially tubular, with an inner diameter slightly greater than the diameter of advancement guide 908 and wire array 902 such that advancement mechanism 932 can snugly fit onto and slide along advancement guide assembly 910. The inner diameter of advancement mechanism 932 is also less than the outer diameter of medical device 110 such that a distal end face 934 of advancement mechanism 932 can contact proximal end 112 of medical device 110 to advance medical device 110 distally.

As shown in FIG. 9, expander 906 is similar to expander 130, except that proximal end 200A of expander 130 is omitted from expander 906. That is, expander 930 includes only transition portion 200C and distal end 200B. As such, a proximal end face 926 is positioned at the proximal end of transition portion 200C. Proximal end face 926 is substantially similar in structure and size to proximal end face 204 discussed above and also includes the opening to guide opening 320 that extends into expander 906. Because expander 906 includes transition portion 200C and distal end 200B, expander 906 also includes wire guides 210 formed thereon. Wire guides 210 terminate at proximal end face 926.

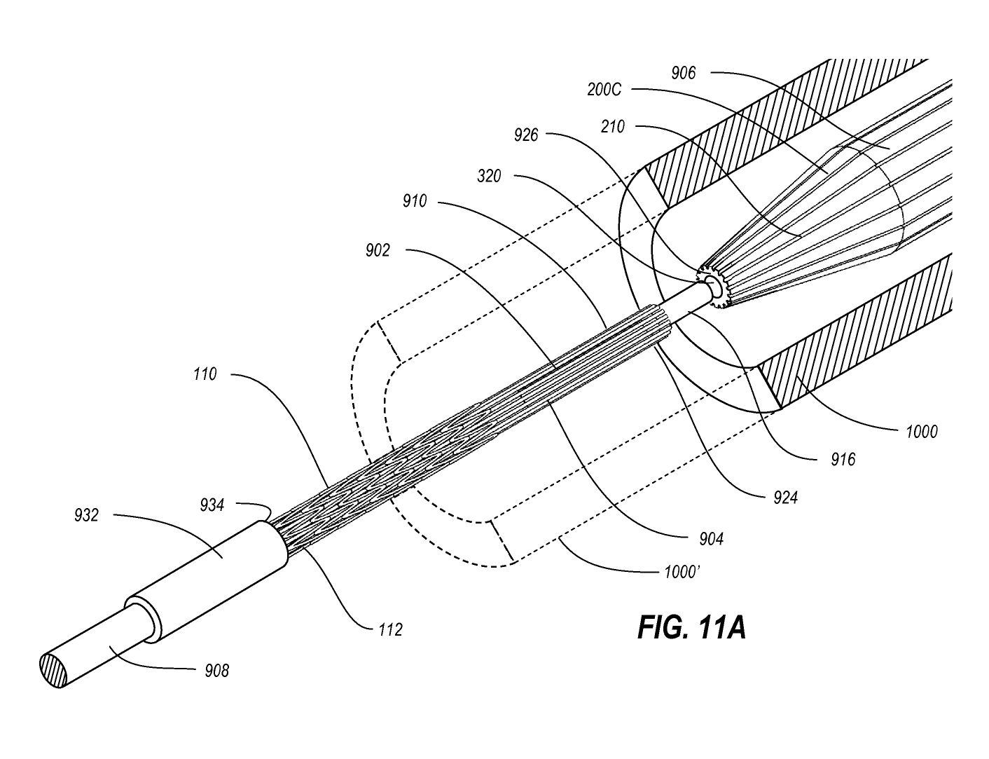

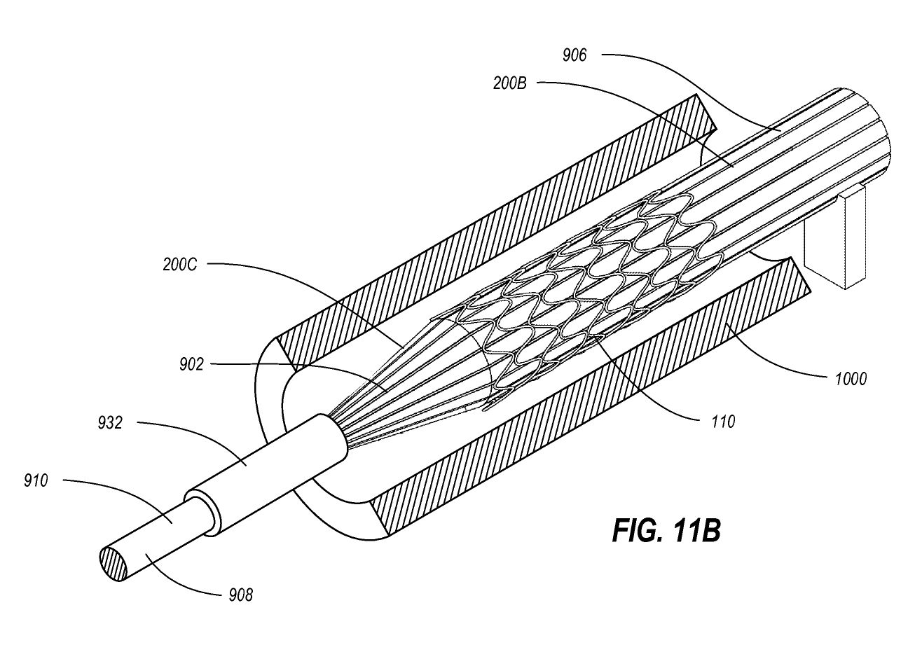

FIGS. 11A-11B illustrate one embodiment of a method of uniformly expanding (and heat setting, if desired) medical device 110 using advancement guide assembly 910. Similar to the method illustrated in FIGS. 8A-8C, a thermal chamber 1000 is used that has an axial length that is substantially the same as or slightly longer or shorter than expander 906, as shown in FIG. 11B, and remains axially aligned with expander 906 during the expansion process. However, because expander 906 is missing proximal end 200A, thermal chamber 1000 can be substantially axially shorter than thermal chamber 800.

Thermal chamber 1000 can be longer or shorter, if desired. For example, thermal chamber 1000 can extend proximally beyond expander 906 (as shown by dashed lines 1000′ in FIG. 11A) to allow medical device 110 to be pre-heated before being advanced onto expander 906.

The process can begin by positioning medical device 110 over wire array 902 and advancing advancement guide assembly 910 distally toward expander 906 so that axial guide 916 aligns with guide opening 320 in end face 926 of transition portion 200C, as shown in FIG. 11A. To position medical device 110 over wire array 902, advancement mechanism 932 can be used. Medical device 110 and advancement mechanism 932 are first positioned onto the proximal end of advancement guide 908. Then, advancement mechanism 932 is advanced distally along advancement guide 908, causing distal end face 934 to contact proximal end 112 of medical device 110 and thereby push medical device 110 distally along advancement guide 908 and onto wires 904 of wiring guide 902. Medical device 110 can be positioned on advancement guide assembly 910 before or after axial guide 916 has been aligned with guide opening 320. If axial guide 916 is not used or is shorter than wiring guide 902, advancement guide assembly 910 can be positioned by aligning wires 904 with wire guides 210 on expander 906.

If desired, axial guide 916 and guide opening 320 can be configured to require rotational alignment therebetween prior to insertion of axial guide 916 so as to better align wires 904 with wire guides 210. In one embodiment, axial guide 916 and guide opening 320 can both have matching non-circular cross sectional shapes. For example, axial guide 916 and guide opening 320 can each have an oval cross section, a polygonal cross section, or some other regular or irregular geometric cross section.

In another embodiment, shown in FIG. 12A, a key, such as radial protrusion 928 can be formed on distal end 914 of axial guide 916 and a mating key, such as notch 930, can be formed on guide opening 320 of expander 906 so that advancement guide 908 can only be inserted into guide opening 320 when the mating keys are rotationally aligned.

By requiring rotational alignment before axial guide 916 can be inserted into guide opening 320, wires 904 can be caused to be aligned with wire guides 210 before wires 904 are advanced, thereby avoiding potential wire advancement issues. It is appreciated that other devices and methods for rotational alignment of advancement guide assembly 910 and expander 906 can alternatively be used.

For example, an external alignment mechanism can be used to ensure that advancement guide assembly 910 and expander 906 are rotationally aligned. In one embodiment, advancement guide 908 and/or expander 906 can include one or more alignment engagers which are engaged by corresponding external alignment devices to align the two devices. Each external alignment device can comprise a structure that mates with the alignment engager that is used and that, when mated, can cause the advancement guide 908 and expander 906 to be rotationally aligned and secured with respect to each other.

For example, as shown in the cross sectional view of FIG. 12B, advancement guide 908 can have a pair of flat spots 940 on either side thereof. A pair of clamp arms 942 can be positioned on both sides of advancement guide 908 so as to be aligned with flat spots 940. Each clamp arm 942 can have a clamping surface 944 that includes a flat section 946 corresponding to flat spot 940. As such, when each clamp arm 942 is brought toward each other, as indicated by arrows 948, flat sections 946 can press against flat spots 940 to rotationally align advancement guide 908. Other alignment engagers can be used, such as a bore, a channel, a flange, or any other engager that can be engaged by corresponding external alignment devices.

Returning to FIG. 11A, advancement guide assembly 910 can be further advanced to cause axial guide 916 to be received within guide opening 320 and distal ends 924 of wires 904 to be received on wire guides 210 of transition portion 200C of expander 906. Further distal advancement of advancement guide assembly 910 causes axial guide 916 to continue through guide opening 320 and wires 904 to slide distally along wire guides 210 until medical device 110 becomes positioned adjacent the proximal end 926 of expander 906. If thermal chamber 1000 extends proximally beyond expander 906, such as shown in dashed lines 1000′, medical device 110 can remain positioned adjacent the proximal end 926 of expander 906 for a pre-determined period of time to be pre-heated by thermal chamber 1000.

Further advancement of advancement guide assembly 910 can cause wire array 902 and medical device 110 to be advanced distally on expander 906 to uniformly expand and heat-set medical device 110 in a similar manner to the methods discussed above. That is, further distal advancement of advancement guide assembly 910 causes wires 904 of wire array 902 to advance distally along wire guides 210 in transition portion 200C and distal end 200B. This causes medical device 110 to also be advanced distally on expansion member/expander 902, and to expand as medical device 110 passes over transition portion 200C to the final expanded configuration when positioned on distal end 200B, as shown in FIG. 11B.

By integrating the wire array, axial guide, and advancement guide into a single advancement guide assembly, several advantages can be realized. For example, because the wire array, axial guide, and advancement guide are all rigidly attached, there is no possibility of the wires of the wire array binding within the wire guides or otherwise not advancing when the advancement guide is advanced. Furthermore, because the wires are rigidly attached to the advancement guide, the advancement guide assembly can be configured so that the wires will better align with the wire guides on the expansion member/expander when in use. For example, as discussed above, mating keys can be formed on the axial guide and the guide opening of the expansion member/expander to force the wires to be axially aligned with the wire guides before the wires can be advanced. Other advantages may also be realized.

In the methods discussed above, medical device 110 and wire arrays 120 and 902 are described as moving distally to engage expansion member/expanders 130 and 906 and to expand medical device 110. However, it is appreciated that this movement is relative. As such, the axial movement can be accomplished by any of the following: i) the medical device and wire array can move distally while the expansion member/expander remains axially stationary, ii) the expansion member/expander can move proximally while the medical device and wire array remain axially stationary, or iii) the expansion member/expander, the medical device, and wire array can all move axially, the medical device and wire array moving in the opposite axial direction as the expansion member/expander.

In one embodiment, medical device 110 can include a material made from any of a variety of known suitable materials, such as a shape-memory material (“SMM”) or superelastic material. For example, the SMM can be shaped in a manner that allows for restriction to induce a substantially tubular, linear orientation while within a delivery shaft (e.g., delivery catheter or encircling an expandable member), but can automatically retain the memory shape of the medical device once extended from the delivery shaft. SMMs have a shape-memory effect in which they can be made to remember a particular shape. Once a shape has been remembered, the SMM may be bent out of shape or deformed and then returned to its original shape by unloading from strain or heating. SMMs can be shape-memory alloys (“SMA”) or superelastic metals comprised of metal alloys, or shape-memory plastics (“SMP”) comprised of polymers.

An SMA can have any non-characteristic initial shape that can then be configured into a memory shape by heating the SMA and conforming the SMA into the desired memory shape. After the SMA is cooled, the desired memory shape can be retained. This allows the SMA to be bent, straightened, compacted, and placed into various contortions by the application of requisite forces; however, after the forces are released, the SMA can be capable of returning to the memory shape. Examples of SMAs that can be used include, but are not limited to: copper-zinc-aluminum; copper-aluminum-nickel; nickel-titanium (“NiTi”) alloys known as nitinol; and cobalt-chromium-nickel alloys or cobalt-chromium-nickel-molybdenum alloys known as elgiloy. The nitinol and elgiloy alloys can be more expensive, but have superior mechanical characteristics in comparison with the copper-based SMAs. The temperatures at which the SMA changes its crystallographic structure are characteristic of the alloy, and can be tuned by varying the elemental ratios.

For example, the primary material of the medical device 110 can be of a NiTi alloy that forms superelastic nitinol. Nitinol materials can be trained to remember a certain shape, straightened in a shaft, catheter, or other tube, and then released from the catheter or tube to return to its trained shape. Also, additional materials can be added to the nitinol depending on the desired characteristic.

An SMP is a shape-memory polymer or plastic that can be fashioned into medical device 110 in accordance with the present invention. When an SMP encounters a temperature above the lowest melting point of the individual polymers, the blend makes a transition to a rubbery state. The elastic modulus can change more than two orders of magnitude across the transition temperature (“Tₜᵣ”). As such, an SMP can be formed into a desired shape of medical device 110 by heating the SMP above the Tₜᵣ, fixing the SMP into the new shape, and cooling the material below Tₜᵣ. The SMP can then be arranged into a temporary shape by force and then resume the memory shape after heating and following removal of the force. Examples of SMPs that can be used include, but are not limited to: biodegradable polymers, such as oligo(ε-caprolactone)diol, oligo(ρ-dioxanone)diol, and non-biodegradable polymers such as, polynorborene, polyisoprene, styrene butadiene, polyurethane-based materials, vinyl acetate-polyester-based compounds, and others yet to be determined. As such, any SMP can be used in accordance with the present invention.

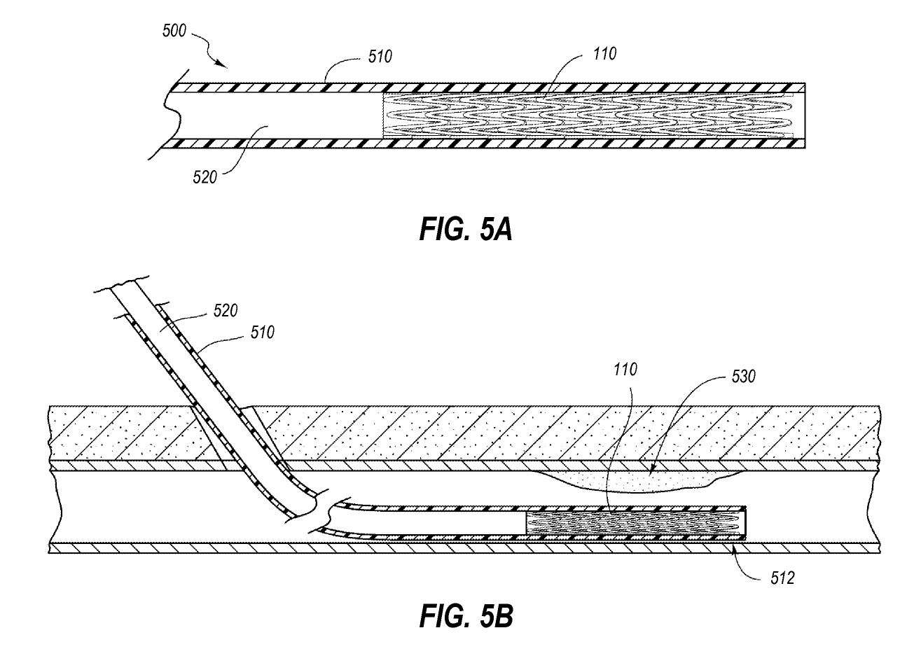

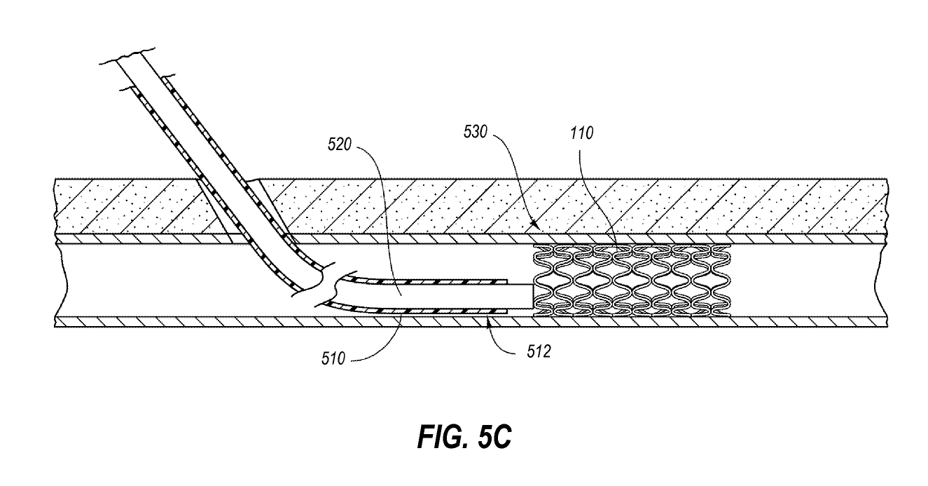

FIGS. 5A-5C illustrate one embodiment of a method for deploying a medical device that has been expanded using any of the methods and devices discussed herein. FIG. 5A illustrates medical device 110 positioned within a deployment device 500 that can include an outer housing 510 and an inner portion 520 positioned within outer housing 510. Inner portion 520 can be operatively associated with an actuation assembly (not shown) to advance inner portion 520 relative to outer housing 510. In at least one embodiment, the deployment method begins by positioning medical device 110 within outer housing 510. Medical device 110 can be positioned within outer housing 510 in any suitable manner, such as through the use of a crimping device or other device that moves medical device 110 from the expanded state to the pre-deployed state shown in FIG. 5A.

After medical device 110 is positioned within outer housing 510, a distal end 512 of outer housing 510 can be positioned at a deployment site 530, as shown in FIG. 5B. With the outer housing 510 in position at the deployment site 530, inner portion 520 can be advanced distally relative to outer housing 510 to urge medical device 110 from distal end 512 of outer housing 510.

In alternative embodiments, medical device 110 can be constrained by a thin housing or sheath. Instead of urging the medical device from within outer housing 510, the thin housing or sheath can be pulled from medical device 110. At the same time, deployment device 500 can be withdrawn and medical device 110 can expand as the thin housing or sheath is removed.

Deployment of medical device 110 from the housing, whether using outer housing 510 or a thin housing, can be accomplished through one or more of: advancing a portion of deployment device 500 (e.g., inner portion 520), withdrawing a portion of deployment device 500 (e.g., outer housing 510), and advancing a portion of medical device 100, whether simultaneously or otherwise. One of skill in the art can appreciate that other known deployment devices and configurations can be used to deploy medical device 110.

In at least one embodiment, when medical device 110 is urged from distal end 512 of deployment device 500, medical device 110 is no longer constrained and can expand towards its expanded state, as illustrated in FIG. 5C. In this manner, medical device 110 can be deployed at deployment site 530.

As previously discussed, the method for forming medical device 110 can reduce localized friction or other factors to provide uniform expansion of medical device 110. Uniform expansion of medical device 110 in turn can allow medical device 110 to be deployed in the intended manner.

While various configurations have been described that include expanders that are self expanding, it will be appreciated that expanders can also be used that require separate expansion mechanisms to become expanded.

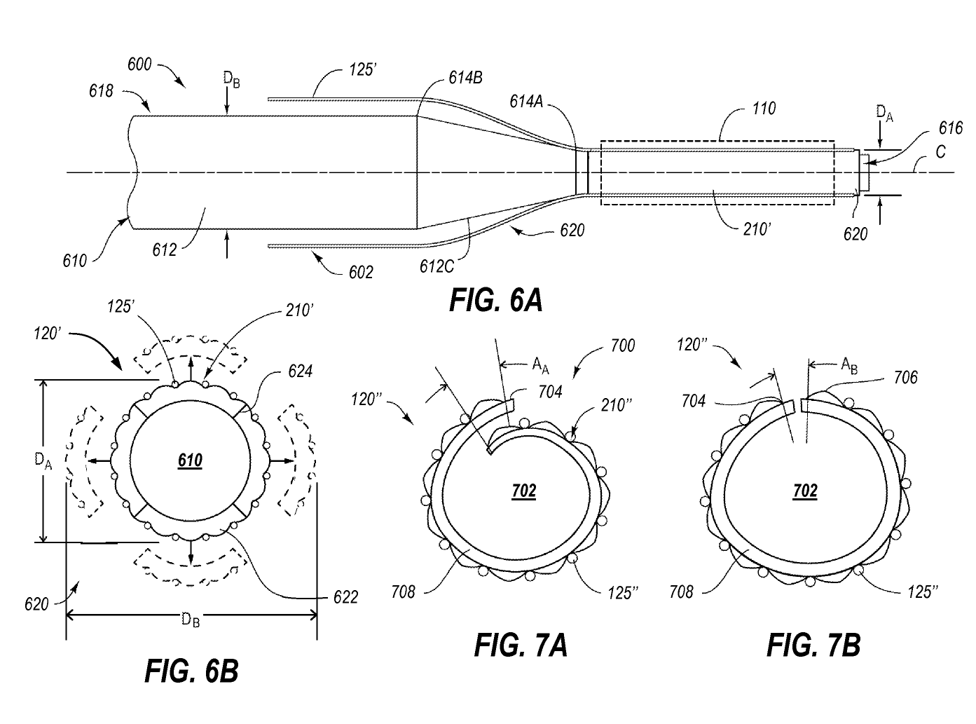

For example, FIG. 6A illustrates an expansion system 600 for expanding medical device 110 that includes an expansion mechanism 610 and an expander 620 positionable upon expansion mechanism 610. The expander 620 can be configured to receive and support a wire array 120′, the cooperation of wire array 120′ and expander 620 being usable to expand medical device 110. For simplicity, a portion of expansion mechanism 610 is illustrated as being received within expander 620 and a number of wires 125′ of wire array 120′ have been omitted in FIG. 6A.

Expansion mechanism 610, illustrated in FIG. 6A, generally includes a body 612 having a proximal end 616 and a distal end 618. Body 612 can have a first diameter DA at proximal end 616 that transitions to a second diameter DB that is greater than first diameter DA. In at least one embodiment, the second diameter DB is at distal end 618, though it will be appreciated that the first diameter DA can transition to any number of varying diameters at any number of locations between proximal end 616 and distal end 618.

In the illustrated embodiment, body 612 transitions from the first diameter DA to the second diameter DB at a transition portion 612C. Transition portion 612C can include a tapered or ramped profile with a shoulder portion 614A associated with proximal end 616 and a shoulder portion 614B associated with distal end 618. It will be appreciated that transition portion 612C and shoulder portions 614A, 614B can have any profile and that any number of transition portions can be provided. Expansion mechanism 610, expander 620, and medical device 110 will be described with common central axis C.

Expander 620 can include a number of segmented portions 622, illustrated in FIG. 6B, separated by slots 624. Segmented portions 622 are configured to interface with the inside diameter of medical device 110, illustrated in phantom in FIG. 6A. In at least one embodiment, segmented portions 622 have wire guides 210′ defined therein that are configured to at least receive a wire array 120′. Segmented portions 622 are configured to interface with the expansion mechanism 610 and to outwardly move as the diameters of the expansion mechanism 610 increase. Wire guides 210′ are defined in segmented portions 622 and are aligned generally parallel to central axis C.

As previously discussed, segmented portions 622 can be supported by expansion mechanism 610. In particular, FIG. 6B illustrates segmented portions 622 positioned over proximal end 616 of expansion mechanism 610. In this position, segmented portions 622 are separated by approximately the distance DA. Expansion mechanism 610 can be advanced axially relative to expander 620 to move first transition portion 612C and then distal end 618 into engagement with expander 620. This is similar to the axial motion of body 200 relative to medical device 110 in FIGS. 4A-4C.

As expander 620 moves into engagement with transition portion 612C and distal end 618, segmented portions 622 and wires 125′ move radially outward, in the direction of the arrows illustrated in FIG. 6B, resulting in a separation of approximately DB, as illustrated in phantom in FIG. 6B. The radially outward movement of segment portions 622 and wires 125′ uniformly expands medical device 110 in a similar manner as described above. The expanded medical device 110 can then be deployed by positioning the medical device in a deployment device as described above.

Generally, expander 620 and/or the expansion mechanism 610 can be fabricated from a variety of different materials. By way of example, expander 620 and/or expansion mechanism 610 can be made from metals, alloys, plastics, polymers, composites, ceramics, quartz, glass, combinations thereof, or other materials, as desired, based upon the particular medical device being formed and the temperatures and/or pressures that expander 620 and/or expansion mechanism 610 are to withstand during the manufacture of the medical device.

In one embodiment, expander 620 and/or expansion mechanism 610 can be fabricated from stainless steel or nitinol. In another embodiment, the materials can withstand a temperature from about 250° C. to about 600° C., from about 250° C. to about 650° C., from about 300° C. to about 600° C., from about 300° C. to about 550° C., from about 450° C. to about 600° C., or some other range known to one skilled in the art in view of the teaching contained herein.

FIGS. 7A-7B illustrate another embodiment of an expander 700. Expander 700 can include a rolled configuration in which expander 700 includes a first end 704, a second end 706 and a central portion 708. Wire guides 210″ can be formed in expander 700 between first end 704 and second end 706 and can receive wires 125″. In such a configuration, first end 704 and second end 706 are separated by at least one angular separation and central portion 708 is curved to define a central lumen 702. In such a configuration angular separation between first end 704 and second end 706 can be increased. In particular, overlap between first end 704 and second end 706 can be described as negative angular separation while a gap between first end 704 and second end 706 can be described as positive angular separation.

Accordingly, a negative angular separation AA is shown in FIG. 7A in which first end 704 and second end 706 overlap. The angular separation AA shown in FIG. 7A can be established when expander 700 is in engagement with a proximal end of an expansion mechanism, such as expansion mechanism 610 described above, upon insertion of expansion mechanism 610 within central lumen 702. Axial movement of expansion mechanism 610 can expand expander 700 to change the angular separation between first end 704 and second end 706 to a positive angular separation AB shown in FIG. 7B. A wire array 120″, similar to the other wire arrays described herein, can be positioned in wire guides 210″ such that as expander 700 is expanded, wire array 120″ and expander 700 are also expanded to allow expansion of a medical device as described above.

Generally, expander 700 can be fabricated from a variety of different materials. By way of example, expander 700 can be made from metals, alloys, plastics, polymers, composites, combinations thereof, or other materials, as desired, based upon the particular medical device being formed and the temperatures and/or pressures that expander 700 is to withstand during medical device manufacture. In one embodiment, expander 700 can be fabricated from stainless steel or nitinol. In one embodiment, the materials can withstand a temperature from about 300° C. to about 600° C.

While one type of expansion mechanism has been provided for expanding expanders 620 and 700, it will be appreciated that other types of expansion mechanisms can be used in a process in which a medical device is expanded with a wire array.

As noted above, although the embodiments discussed herein employ wires as the transport mechanisms, it is appreciated that other types of transport mechanisms can alternatively be used according to the present invention. For example, strips, ribbons, yarns, threads, rods, or other structures can be used as the transport mechanisms, as long as those structures have the desired strength and rigidity, with associated flexibility and resiliency to allow the structure to i) provide a bearing-type surface for the medical device, ii) separate at least a portion of the medical device from an expander or expander mechanism during a manufacturing process, or iii) otherwise perform other functions described or identified from the description contained herein.

The present invention may be embodied in other specific forms without departing from its spirit or essential characteristics. For example, slight modifications of the mandrel are contemplated and possible and still be within the spirit of the present invention and the scope of the claims. The scope of the invention is, therefore, indicated by the appended claims rather than by the foregoing description.

What is claimed is:

1. A method of manufacturing a medical device, the method comprising:
forming a medical device from a tube having a first diameter;
uniformly expanding the medical device from the first diameter to a second diameter at which the medical device can be left within a body vessel, the medical device being expanded from the first diameter to the second diameter while being continuously positioned on an expander; and
heat setting the expanded medical device at the second diameter while the medical device is positioned on the expander.

2. The method of claim 1, wherein heat setting the expanded medical device comprises maintaining the expanded medical device at the second diameter on the expander for a predetermined period of time while the expander is maintained at a predetermined heat-setting temperature.

3. The method of claim 2, wherein the expander is positioned within a thermal chamber that maintains the expander at the predetermined heat-setting temperature during the steps of uniformly expanding the medical device and heat setting the expanded medical device.

4. The method of claim 1, wherein the medical device is comprised of a shape-memory material.

5. The method of claim 1, wherein the medical device is physically separated from the expander when the medical device is positioned on the expander.

6. The method of claim 1, further comprising:
preheating the medical device within a thermal chamber before uniformly expanding the medical device; and
maintaining heat on the medical device within the thermal chamber during radial expansion of the medical device.

7. A method of manufacturing a medical device, the method comprising:
forming a medical device from a tube having a first diameter;
supporting the medical device on an expander, the medical device being supported by and spaced apart from an outer surface of the expander;
uniformly expanding the medical device from the first diameter to a second diameter at which the medical device can be left within a body vessel, the medical device being expanded from the first diameter to the second diameter while being continuously positioned on the expander and separated from the outer surface of the expander; and
heat setting the expanded medical device at the second diameter while the medical device is positioned on the expander.

8. The method of claim 7, wherein supporting the medical device on the expander comprises supporting the medical device on segmented portions of the expander.

9. The method of claim 7, further comprising positioning segmented portions of the expander on an expansion mechanism before uniformly expanding the medical device.

10. The method of claim 7, further comprising positioning longitudinal elements between the medical device and the expander, the longitudinal elements supporting the medical device.

11. The method of claim 7, wherein supporting the medical device on the expander comprises supporting the medical device on a volute expander.

12. The method of claim 11, wherein uniformly expanding the medical device further comprises unrolling the expander from a first diameter with a first end and a second end of the expander having a first angular separation to a second diameter with the first end and the second end having a second angular separation.

13. The method of claim of claim 12, wherein the first angular separation is a negative angular separation.

14. The method of claim 12, wherein the second angular separation is a positive angular separation.

15. The method of claim 7, further comprising aligning a plurality of longitudinal structures disposed between the medical device and the expander within a plurality of transport guides formed on a surface of the expander.

16. The method of claim 15, wherein aligning the plurality of longitudinal structures comprises aligning an internally positioned alignment mechanism with a lumen of the expander.

17. The method of claim 15, wherein aligning the plurality of longitudinal structures comprises aligning a pair of clamp arms with an advancement guide configured to rotationally align with the expander.

18. A method of manufacturing a medical device, the method comprising:
positioning a medical device on a transport assembly having a plurality of transport mechanisms, the transport mechanisms being arranged generally parallel to a central longitudinal axis;
positioning a portion of the transport assembly on an expander so that the medical device becomes positioned radially over the expander;
radially expanding the medical device with the expander while the medical device is positioned on the transport assembly; and
heat setting the expanded medical device while the medical device is positioned on the expander, the acts of radially expanding the medical device and heat setting the expanded medical device being performed while the medical device is positioned in a heated thermal chamber.

19. The method of claim 18, wherein positioning the portion of the transport assembly on the expander comprises:
positioning ends of the transport mechanisms of the transport assembly in transport guides formed on the expander; and
moving the transport assembly longitudinally with respect to the expander so that the transport mechanisms slide along the transport guides until the medical device is positioned radially over the expander.

20. The method of claim 19, wherein positioning the portion of the transport assembly on the expander further comprises:
inserting an axial guide into a guide opening extending longitudinally through the expander, the transport assembly being attached to the axial guide so as to move therewith, and
wherein the steps of positioning the ends of the transport mechanisms in the transport guides and moving the transport assembly longitudinally are accomplished by moving the axial guide along the guide opening.

21. The method of claim 18, wherein radially expanding the medical device with the expander comprises moving the medical device relative to the expander from a first position on a first portion of the expander to a second position on a second portion of the expander, the first and second portions of the expander respectively having first and second cross sectional areas, the second cross sectional area being greater than the first cross sectional area, the medical device being radially expanded as the medical device moves from the first position to the second position.
